(19) United States
(12) Patent Application Publication
(10) Pub. No.: US 2023/0368876 A1
(43) Pub. Date: Nov. 16, 2023

(54) MEDICAL SUPPORT SYSTEM

(71) Applicant: Canon Medical Systems Corporation, Otawara-shi (JP)

(72) Inventors: Nao WARIISHI, Nasushiobara (JP); Hiroki SAITO, Oyama (JP)

(73) Assignee: Canon Medical Systems Corporation, Otawara-shi (JP)

(21) Appl. No.: 18/307,122

(22) Filed: Apr. 26, 2023

(30) Foreign Application Priority Data
May 13, 2022 (JP) 2022-079532

(51) Int. Cl. G16H 10/60; G16H 50/20

(52) U.S. Cl. CPC G16H 10/60; G16H 50/20

(57) ABSTRACT

A medical support system includes a processing circuit configured to: acquire medical data on a patient over time; cause a display to display a display screen on which one or more measurement values in the medical data are displayed; detect an alert value that satisfies a determination condition from the one or more measurement values included in the medical data every time the medical data is acquired; and highlight an area in which the alert value is displayed on the display screen, and cause the display screen to display information on the determination condition for all detected alert values.

### CROSS-REFERENCE TO RELATED APPLICATIONS

This application is based upon and claims the benefit of priority from Japanese Patent Application No. 2022-079532, filed May 13, 2022, the entire contents of which are incorporated herein by reference.

### FIELD

Embodiments described herein relate generally to a medical support system.

### BACKGROUND

Conventionally, a medical information integrative viewer for use in emergency medical care gives an alert (warning) in a case where medical data such as results of laboratory tests, vital signs, etc. satisfies a predetermined condition. For example, in response to a user pressing a button, if a value of medical data currently displayed on a screen exceeds a specific threshold value, a corresponding portion is highlighted. In such a case, execution of alert processing is triggered by the button being pressed. Thus, the alert processing cannot be executed in real time with respect to medical data to obtain. Furthermore, since only the data currently displayed on the screen is highlighted, it is impossible to know whether or not an alert has occurred with respect to medical data not currently displayed on the screen. To confirm whether or not an alert has occurred with respect to medical data not currently displayed on the screen, multiple operations on the display screen are required.

### BRIEF DESCRIPTION OF THE DRAWINGS

FIG. 1 is a diagram showing an example of a configuration of a medical support system according to a first embodiment.

FIG. 2 is a diagram showing an example of a configuration of a server device according to the first embodiment.

FIG. 3 is a diagram showing an example of a configuration of a client device according to the first embodiment.

FIG. 4 is a flowchart showing an example of a processing procedure of emergency medical care support processing by the medical support system according to the first embodiment.

FIG. 5 is a diagram showing an example of a display screen displayed by the medical support system according to the first embodiment.

FIG. 6 is a flowchart showing an example of a processing procedure of emergency medical care support processing by a medical support system according to a modification of the first embodiment.

FIG. 7 is a diagram showing an example of a display screen displayed by the medical support system according to the modification of the first embodiment.

FIG. 8 is a flowchart showing an example of a processing procedure of emergency medical care support processing by the medical support system according to a second embodiment.

FIG. 9 is a diagram showing an example of a display screen displayed by the medical support system according to the second embodiment.

FIG. 10 is a flowchart showing an example of a processing procedure of emergency medical care support processing by a medical support system according to a modification of the second embodiment.

FIG. 11 is a diagram showing an example of a display screen displayed by the medical support system according to the modification of the second embodiment.

FIG. 12 is a flowchart showing an example of a processing procedure of emergency medical care support processing by a medical support system according to a third embodiment.

FIG. 13 is a diagram showing an example of a display screen displayed by the medical support system according to the third embodiment.

FIG. 14 is a diagram showing an example of a display screen displayed by the medical support system according to the modification of the third embodiment.

### DETAILED DESCRIPTION

In general, according to one embodiment, a medical support system includes a processing circuit configured to: acquire medical data on a patient over time; cause a display to display a display screen on which one or more measurement values in the medical data are displayed; detect an alert value that satisfies a determination condition from the one or more measurement values included in the medical data every time the medical data is acquired; and highlight an area in which the alert value is displayed on the display screen, and cause the display screen to display information on the determination condition for all detected alert values.

Hereinafter, embodiments of a medical support system will be described in detail with reference to the accompanying drawings. In the following description, structural elements having approximately the same function and configuration will be assigned the same reference symbol, and a repeat description will be given only where necessary.

### First Embodiment

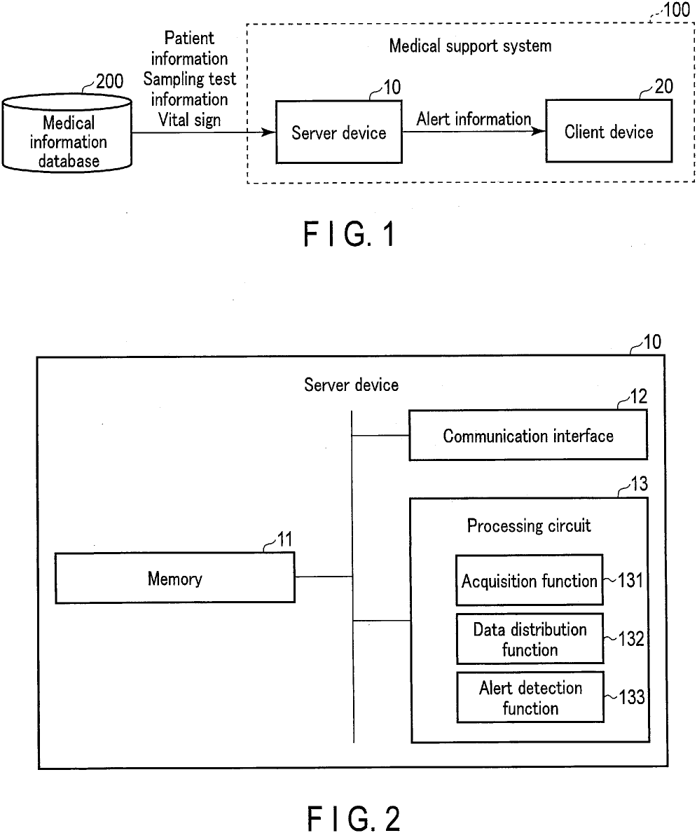

FIG. 1 is a diagram showing a configuration of a medical support system 100. The medical support system 100 supports and manages medical care for emergency patients. The medical support system 100 is connected to a medical information database 200 via a network. The medical support system 100 is capable of transmitting and receiving various types of information to and from the medical information database 200 via the network.

The network is, for example, a Local Area Network (LAN). The connection to the network may be established in either a wired or wireless manner. Furthermore, as long as security is ensured by means of a Virtual Private Network (VPN), etc., a line to be connected is not necessarily a LAN. A connection to the Internet or other public communication network may be established.

The medical information database 200 stores and manages medical data on emergency patients. The medical data includes, for example, patient information, sampling test information, and vital information.

The patient information is information on an emergency patient. The patient information includes information on a patient’s name, gender, age, nationality, medical history, and a test, a result of a medical examination, a medical image, etc.

The sampling test information is information on a sampling test. The sampling test is, for example, a blood test. The sampling test information includes a sampling test item name, a test method, a test date and time, a test result, etc. A sampling test result includes a hemoglobin concentration (ctHb), oxygen saturation (SpO2), a P/F ratio, a lactate level (cLac), etc.

The vital information is information on a measurement result of a vital sign, and includes, for example, a measurement value of the vital sign. Measurement values of a vital sign include a heart rate (HR), an upper blood pressure value (ABP S), an average blood pressure value (ABP M), a lower blood pressure value (ABP D), etc. The measurement values of a vital sign may include other given measurement values.

The medical information database 200 is connected, via a network, to a patient information system (not shown) that manages the patient information and the sampling test information, a vital sign manager (not shown) that manages a vital sign transmitted from a vital sign measurer attached to a patient, etc. The medical information database 200 acquires the patient information and the sampling test information from the patient information system every several tens of minutes, for example. The medical information database 200 further acquires the vital information from the vital sign manager every second, for example. An interval at which the sampling test information is acquired is set to, for example, an interval at which the sampling test result is updated. Since the vital sign is constantly measured, the vital information is preferably acquired every few seconds.

The medical support system 100 includes a server device 10 and a client device 20. The server device 10 is, for example, a server installed in an emergency medical facility. For example, the server device 10 receives patient’s medical data from the medical information database 200, and performs predetermined data processing on the received medical data. Furthermore, the server device 10 detects a measurement value indicating an abnormal value (hereinafter referred to as an alert value) by executing alert processing based on medical data, and transmits alert information including a detection result of the alert value, to the client device 20. The client device 20 is, for example, a terminal device installed in an emergency room. The client device 20 receives the data-processed medical data and alert information from the server device 10, and highlights the display screen corresponding to the medical data based on the alert information.

Next, a configuration of the server device 10 will be described.

FIG. 2 is a diagram showing the configuration of the server device 10. The server device 10 has a memory 11, a communication interface 12, a display 23, an input interface 24, and a processing circuit 13. Hereinafter, the server device 10 will be described as a single device that executes a plurality of functions; however, these functions may be executed by separate devices. For example, the functions executed by the server device 10 may be mounted in different console devices or work station devices in a distributed manner.

The memory 11 is a storage device such as a hard disk drive (HDD), a solid state drive (SSD), or an integrated circuit which stores various types of information. Other than the HDD and SSD, the memory 11 may be a portable storage medium such as a compact disc (CD), a digital versatile disc (DVD), a flash memory, etc. Alternatively, the memory 11 may be a driver that writes and reads various types of information to and from a semiconductor memory such as a flash memory, a random-access memory (RAM), etc. The storage area of the memory 11 may be in the server device 10 or in an external storage device connected via a network.

The memory 11 stores a program to be executed by the processing circuit 13, various types of data to be used for the processing by the processing circuit 13, etc. Such a program may be installed in advance in a computer from a network or a non-transitory computer-readable storage medium, for example, so that the program will cause the computer to implement the respective functions of the processing circuit 13. Various types of data handled in this disclosure are typically digital data. The memory 11 is one example of a storage.

The communication interface 12 is a network interface configured to control communications between the client device 20, the medical information database 200, and an external device via a network.

The processing circuit 13 controls the overall operation of the server device 10. The processing circuit 13 is a processor configured to, upon calling up and executing a program from the memory 11, implement an acquisition function 131, a data distribution function 132, and an alert detection function 133.

The term “processor” used herein refers to, for example, a central processing unit (CPU) or a graphics processing unit (GPU), or a circuit such as an application-specific integrated circuit (ASIC), a programmable logic device (such as a simple programmable logic device (SPLD)), a complex programmable logic device (CPLD), a field programmable gate array (FPGA)), etc. In the case of the processor being a CPU, for example, the processor realizes a function by reading and executing a program stored in a storage circuit. On the other hand, in the case of the processor being, for example, an ASIC, instead of a program being stored in a storage circuit, a corresponding function is directly incorporated as a logic circuit in the circuit of the processor. Each processor of the present embodiment is not limited to a configuration as a single circuit; a plurality of independent circuits may be combined into one processor to realize the functions of the processor. Furthermore, multiple structural elements shown in FIGS. 1, 2, or 3 may be integrated as one processor to realize their functions. The above description of the “processor” is applicable to the subsequent embodiments and modifications.

FIG. 1 assumes that the single processing circuit 13 realizes the acquisition function 131, the data distribution function 132, and the alert detection function 133; however, the processing circuit may be configured by a combination of a plurality of independent processors, and the functions may be realized by the processors executing the programs. The acquisition function 131, the data distribution function 132, and the alert detection function 133 may be implemented as individual hardware circuits. The above description of each of the functions executed by the processing circuit 13 is applicable to the embodiments and modifications described below.

The description assumes that the server device 10 executes multiple functions with a single console; however, these functions may be implemented by separate devices. For example, the functions of the processing circuit 13 may be mounted in different devices in a distributed manner.

The processing circuit 13 acquires, through the acquisition function 131, the medical data on an emergency patient. The processing circuit 13 acquires, as the medical data, the patient information, the sampling test information, and the vital information. As described above, the patient information and the sampling test information are acquired from the medical information database 200 every several tens of minutes, and the vital information is acquired from the medical information database 200 every second. The processing circuit 13 that realizes the acquisition function 131 is one example of the acquirer.

The processing circuit 13 performs, through the data distribution function 132, predetermined data processing on acquired medical data in a batch and transmits the data-processed medical data to the client device 20 via the communication interface 12. The processing circuit 13 that realizes the data distribution function 132 is one example of a data distributer.

The processing circuit 13 executes, through the alert detection function 133, the alert processing based on the medical data. In execution of the alert processing, every time the medical data is acquired through the acquisition function 131, the processing circuit 13 detects a measurement value (hereinafter referred to as an alert value) that satisfies a determination condition (hereinafter referred to as an alert condition) relating to an alert from measurement values included in the medical data. The alert condition is a condition for determining whether the measurement value is abnormal or not. The alert condition is set for each item of the medical data. The alert condition is a conditional expression representing, for example, a large/small relationship with a threshold value, a range of normal values, etc. The alert condition is prestored in the memory 11, for example. The processing circuit 13 transmits alert information including a detection result of the alert value to the client device 20 via the communication interface 12. The alert information includes, in addition to an alert value, a name of an item for which the alert value has been detected (hereinafter referred to as an alert item), and an alert condition for the alert item. The processing circuit 13 that realizes the alert detection function 133 is one example of the detector.

Next, a configuration of the client device 20 will be described.

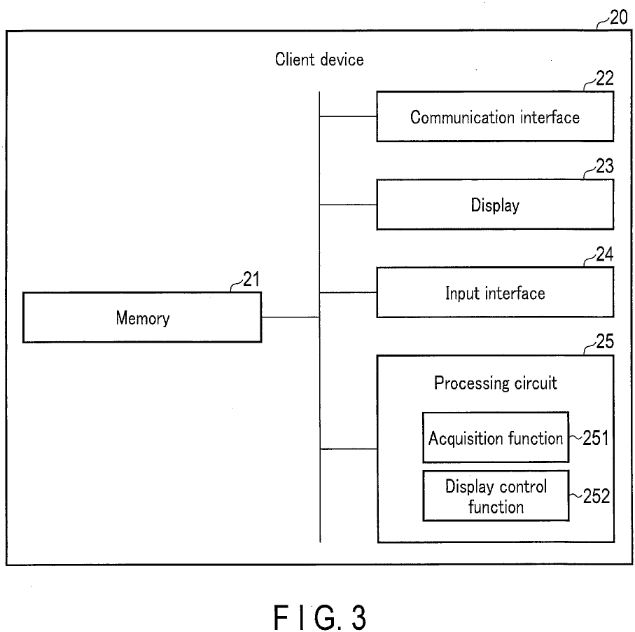

FIG. 3 is a diagram showing the configuration of the client device 20. The client device 20 includes the memory 21, a communication interface 22, the display 23, the input interface 24, and a processing circuit 25. Hereinafter, the client device 20 will be described as a single device that executes a plurality of functions; however, these functions may be executed by separate devices. For example, the functions executed by the client device 20 may be provided in different console devices or work station devices in a distributed manner.

The memory 21 is a storage device such as a hard disk drive (HDD), a solid state drive (SSD), or an integrated circuit which stores various types of information. Other than the HDD and SSD, the memory 21 may be a portable storage medium such as a compact disc (CD), a digital versatile disc (DVD), a flash memory, etc. Alternatively, the memory 21 may be a driver that writes and reads various types of information to and from a semiconductor memory such as a flash memory, a random-access memory (RAM), etc. The storage area of the memory 21 may be in the client device 20 or in an external storage device connected via a network.

The memory 21 stores a program to be executed by the processing circuit 25, various types of data to be used for the processing by the processing circuit 25, etc. Such a program may be installed in advance in a computer from a network or a non-transitory computer-readable storage medium, for example, so that the program will cause the computer to implement the respective functions of the processing circuit 25. Various types of data handled in this disclosure are typically digital data. The memory 21 is another example of the storage.

The communication interface 22 is a network interface configured to control communications between the client device 20, the medical information database 200, and an external device via a network.

The display 23 is configured to display various types of information. For example, the display 23 outputs medical information generated by the processing circuit 25, a graphical user interface (GUI) for receiving various operations from an operator, etc. For example, the display 23 is a liquid crystal display or a cathode ray tube (CRT) display. The display 23 is one example of a displayer.

The input interface 24 receives various input operations from an operator, converts the received input operations into electric signals, and outputs the converted electric signals to the processing circuit 25. For example, the input interface 24 receives input of medical information, input of various command signals, etc., from the operator. The input interface 24 is implemented by a mouse and keyboard for executing various types of processing of the processing circuit 25, a trackball, a switch button, a touch screen in which a display screen and a touch pad are integrated, a non-contact input circuit using an optical sensor, a voice input circuit, etc. The input interface 24 is connected to the processing circuit 25, converts an input operation received from an operator into an electric signal, and outputs the converted electric signal to the control circuit. In this disclosure, the input interface is not limited to one provided with physical operating components such as a mouse and a keyboard. Examples of the input interface include an electric-signal processing circuit that receives an electric signal corresponding to an input operation from an external input device provided separately from the client device, and outputs the received electric signal to the processing circuit 25. The input interface 24 is one example of an inputter.

The processing circuit 25 controls the overall operation of the client device 20. The processing circuit 25 is a processor configured to, upon calling up and executing a program from the memory 21, implement an acquisition function 251 and a display control function 252.

FIG. 1 assumes that the single processing circuit 25 realizes the acquisition function 251 and the display control function 252; however, the processing circuit may be configured by a combination of a plurality of independent processors, and the functions may be realized by the processors executing the programs. The acquisition function 251 and the display control function 252 may be implemented as individual hardware circuits. The above description of the functions executed by the processing circuit 25 is applicable to the embodiments and modifications described below.

The description assumes that the client device 20 executes multiple functions with a single console; however, these functions may be implemented by separate devices. For example, the functions executed by the processing circuit 25 may be provided in different devices in a distributed manner.

The processing circuit 25 acquires, through the acquisition function 251, the medical data on a patient. The processing circuit 25 acquires, as the medical data, the patient information, the sampling test information, and the vital information from the server device 10. The processing circuit 25 acquires, through the acquisition function 251, the alert information from the server device 10. For example, the medical data and the alert information are transmitted from the server device 10 to the client device 20 every time the medical data is updated in the server device 10. The processing circuit 25 that implements the acquisition function 251 is another example of the acquirer.

The processing circuit 25 causes, through the display control function 252, the display 23 to display various types of information. For example, based on the acquired medical data, the processing circuit 25 causes the display 23 to display a display screen on which measurement values in the medical data are displayed. The processing circuit 25 updates the medical data displayed on the display 23 every time new medical data is acquired. The processing circuit 25 that realizes the display control function 252 is an example of the display controller.

Furthermore, through the display control function 252, the processing circuit 25 highlights an area of the display screen in which the alert value is displayed (hereinafter referred to as a warning area). For example, the processing circuit 25 changes a background color in the warning area on the display screen to a conspicuous color such as red.

The processing circuitry 25 causes, through the display control function 252, the display screen to display information on the determination conditions for all of the detected alert values. Specifically, the processing circuit 25 causes the display screen to display information on the alert condition for the alert item. The information on the alert condition includes an alert value, a conditional expression for use as the alert condition, a name of the alert item, a measurement value of the alert item, etc.

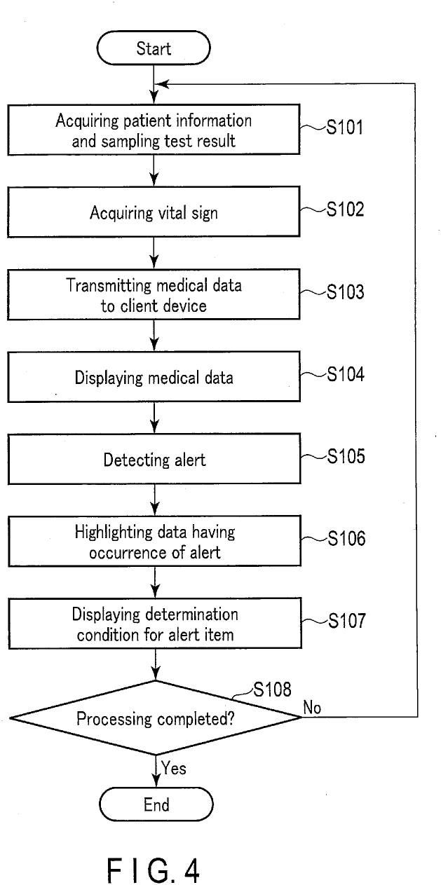

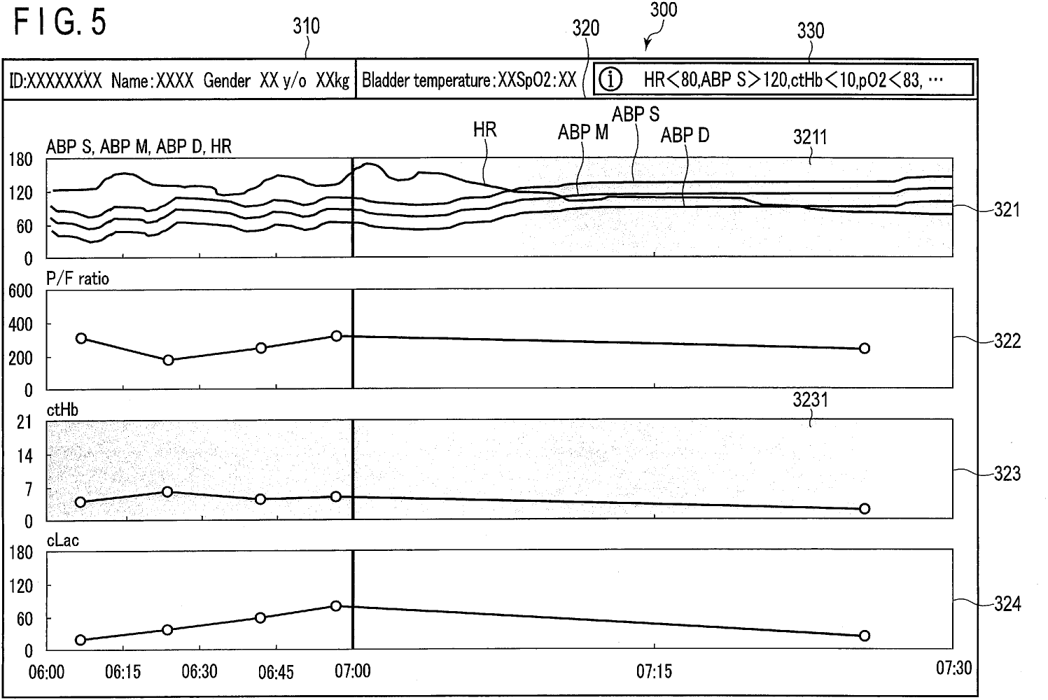

Next, the operation of the emergency medical care support processing executed by the medical support system 100 will be described. The emergency medical care support processing causes the display to display medical data on an emergency patient all the time while he or she undergoes a medical examination. In the case where any one of the measurement values in the medical data satisfies the alert condition, the emergency medical care support processing gives a warning. FIG. 4 is a flowchart showing an example of a processing procedure of emergency medical care support processing. Note that the processing procedure described below is only an example, and each processing can be changed as appropriate where possible. Omission, replacement, and addition of a step in the processing procedure described hereinafter can be made as appropriate, in accordance with an actual situation where the present embodiment is realized.

### (Emergency Medical Care Support Processing) (Step S101)

The processing circuit 13 of the server device 10 acquires, through the acquisition function 131, the patient information on an emergency patient and the sampling test information including a sampling test result from the medical information database 200. For example, the processing circuit 13 acquires the latest patient information and sampling test information from the medical information database 200 every several tens of minutes, and updates information in the memory 11.

### (Step S102)

The processing circuit 13 acquires, through the acquisition function 131, the vital information including a vital sign of an emergency patient from the medical information database 200. For example, the processing circuit 13 acquires the latest vital information from the medical information database 200 every minute, and updates information in the memory 11.

### (Step S103)

The processing circuit 13 transmits, through the data distribution function 132, the medial data on the emergency patient to the client device 20. For example, every time the sampling test information or vital information is updated, the processing circuit 13 transmits the updated medical data to the client device 20.

### (Step S104)

The processing circuit 25 of the client device 20 receives, through the acquisition function 251, the medical data transmitted from the server device 10. Thereafter, the processing circuit 25 causes, through the display control function 252, the display 23 to display the display screen on which the medical data acquired from the server device 10 is displayed.

FIG. 5 is a diagram showing an example of the display screen 300 displayed on the display 23. In the example shown in FIG. 5, the display screen 300 includes a patient information displayer 310, a medical data displayer 320, and an alert information displayer 330.

The patient information displayer 310 displays thereon patient information on an emergency patient to be managed. The patient information displayer 310 displays thereon, for example, a name, a gender, an age, and a weight of the patient.

The medical data displayer 320 displays thereon a plurality of graphs indicating a change over time in measurement values of the medical data on the emergency patient. Each item of the medical data displayed on the medical data displayer 320 is preset. In the example shown in FIG. 5, the medical data displayer 320 includes a first displayer 321 indicating a change over time in a heart rate (HR) and respective blood pressure values (ABP S, ABP M, and ABP D), a second displayer 322 indicating a change over time in a P/F ratio, a third displayer 323 indicating a change over time in a hemoglobin concentration (ctHb), and a fourth displayer 324 indicating a change over time in a lactate level (cLac).

### (Step S105)

The processing circuit 13 of the server device 10 detects, through the alert detection function 133, the alert value from the medical data using the alert condition. At this time, with respect to the medical data acquired through the processing at step S104, the processing circuit 13 detects, as the alert value, the measurement value indicating the abnormal value, using the alert condition preset for each item. The processing circuit 13 transmits, through the data distribution function 132, the alert information including the alert value, the name of the alert item, and the alert condition for the alert item to the client device 20.

### (Step S106)

The processing circuit 25 of the client device 20 receives, through the acquisition function 251, the alert information from the server device 10. The processing circuit 25 highlights, through the display control function 252, the warning area in which the alert value is displayed on the display screen displayed on the display 23.

FIG. 5 shows a case in which alert values include a measurement value of an upper blood pressure (ABP S) and a measurement value of a hemoglobin concentration (ctHb). In the example shown in FIG. 5, the alert condition for the upper blood pressure (ABP S) is a measurement value of the upper blood pressure (ABP S) being greater than 120, and the alert condition for the hemoglobin concentration (ctHb) is a measurement value of the hemoglobin concentration (ctHb) being smaller than 10.

In the example shown in FIG. 5, a warning area 3211 showing a measurement value greater than 120 is highlighted on the first displayer 321, and a warning area 3231 showing a measurement value smaller than 10 is highlighted on the third displayer 323. The warning areas 3211 and 3231 are highlighted by changing their background color to a color different from those of the remaining areas. Alternatively, to highlight the warning areas 3211 and 3231, their background color may be made deeper than those of the remaining areas, or the warning areas 3211 and 3231 may be shaded or enlarged. In addition to highlighting of the warning areas 3211 and 3231, a warning may be given by a sound, etc.

### (Step S107)

The processing circuit 25 displays, through the display control function 252, the alert condition for the alert item.

In one example shown in FIG. 5, the alert information displayer 330 displays thereon a name of an alert item and an alert condition for the alert item. The alert item for which the alert value has been detected is, even if it is an item not displayed on the medical data displayer 320, displayed in the form of a list on the alert information displayer 330.

### (Step S108)

The processing circuit 13 and the processing circuit 25 repeatedly execute the processing from step S101 to step S107 for every predetermined period of time until the treatment on an emergency patient is completed. For example, the processing from step S101 to step S107 is repeatedly executed each time a measurement result of a vital sign is updated every second in the medical information database 200. Furthermore, for example, the processing from step S101 to step S107 is repeatedly executed each time the sampling test information is updated every several tens of minutes in the medical information database 200.

Hereinafter, the effect of the medical support system 100 according to the present embodiment will be described.

The medical support system 100 according to the present embodiment is configured to acquire medical data on a patient over time, cause the display to display the display screen on which measurement values of the medical data are displayed, and detect an alert value from the measurement values included in the medical data. Furthermore, the medical support system 100 is configured to highlight an area in which the alert value is displayed on the display screen, and cause the display screen to display information on the determination conditions for all of the detected alert values. The information on the determination condition for the alert value includes, for example, an alert value, a conditional expression for the alert value, and a name of an alert item.

The configuration described above enables the medical support system 100 according to the present embodiment to execute the alert processing every time the medical data is updated, and updates the display screen based on a detection result of an alert, thereby enabling execution of the alert processing in real time on the acquired medical data. Furthermore, since the alert processing is automatically executed every time the medical data is acquired, a user’s operation load can be reduced. This enables the occurrence of an alert to be efficiently reported in emergency medical care.

Furthermore, a user can be warned of the occurrence of an alert by highlighting a warning area in which a corresponding alert value is displayed. Even in a case where an alert occurs for medical data that is not displayed on the display screen, information on a determination condition for a corresponding alert value is displayed in form of a list, so that a user can easily confirm, without having the burden of performing operations, whether or not an alert has occurred for the medical data not displayed on the display screen.

### Modification of First Embodiment

A modification of the first embodiment will be described. The present modification is a modification of the configuration of the first embodiment as will be described below. Descriptions of the configurations, operations, and advantageous effects similar to those of the first embodiment will be omitted.

In the present modification, the processing circuit 13 of the server device 10 determines, through the alert detection function 133, a trend of change in an alert item. For example, in a case where a measurement value of an alert item continuously rises, this alert item is determined to be on an upward trend. For example, in a case where a measurement value of an alert item continuously falls, this alert item is determined to be on a downward trend. For example, in a case where the amount of change in measurement value during a predetermined period of time is greater than a predetermined value, a trend of change in the corresponding item is determined to be a sharp rise or a sharp drop. For example, in a case where a measurement value of an alert item repeatedly rises and falls, a trend of change is determined to be fluctuating. The processing circuit 13 transmits alert information including a trend of change in an alert item to the client device 20 via the communication interface 12.

The processing circuit 25 of the client device 20 acquires, through the acquisition function 251, the alert information including a trend of change in an alert item from the client device 20.

The processing circuit 25 causes, through the display control function 252, the display 23 to display the trend of change in an alert value. Specifically, the processing circuit 25 causes the display 23 to display the trend of change in an alert item in addition to the information on the determination condition for the alert value.

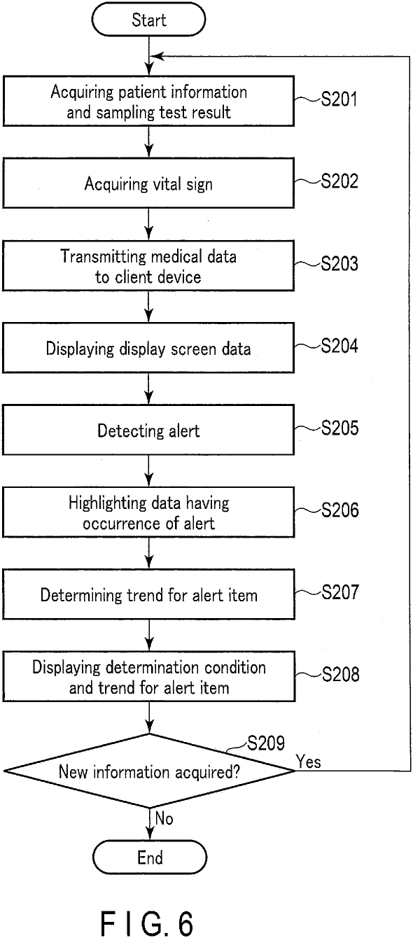

Next, the operation of the emergency medical care support processing executed by the medical support system 100 according to the present modification will be described. FIG. 6 is a flowchart showing an example of a processing procedure of emergency medical care support processing. Since the processing at steps S201 to S206 and S209 is the same as the processing at steps S101 to S106 and S108 in FIG. 4, descriptions thereof are omitted.

### (Emergency Medical Care Support Processing) (Step S207)

The processing circuit 13 of the server device 10 determines, through the alert detection function 133, a trend of change in an alert item using medical data of the alert item. A determination result is transmitted to the client device 20.

### (Step S208)

The processing circuit 25 of the client device 20 receives, through the acquisition function 251, the determination result of the trend of change in an alert item from the server device 10. The processing circuit 25 causes, through the display control function 252, the display 23 to display the trend of change in the alert item in addition to an alert condition for the alert item.

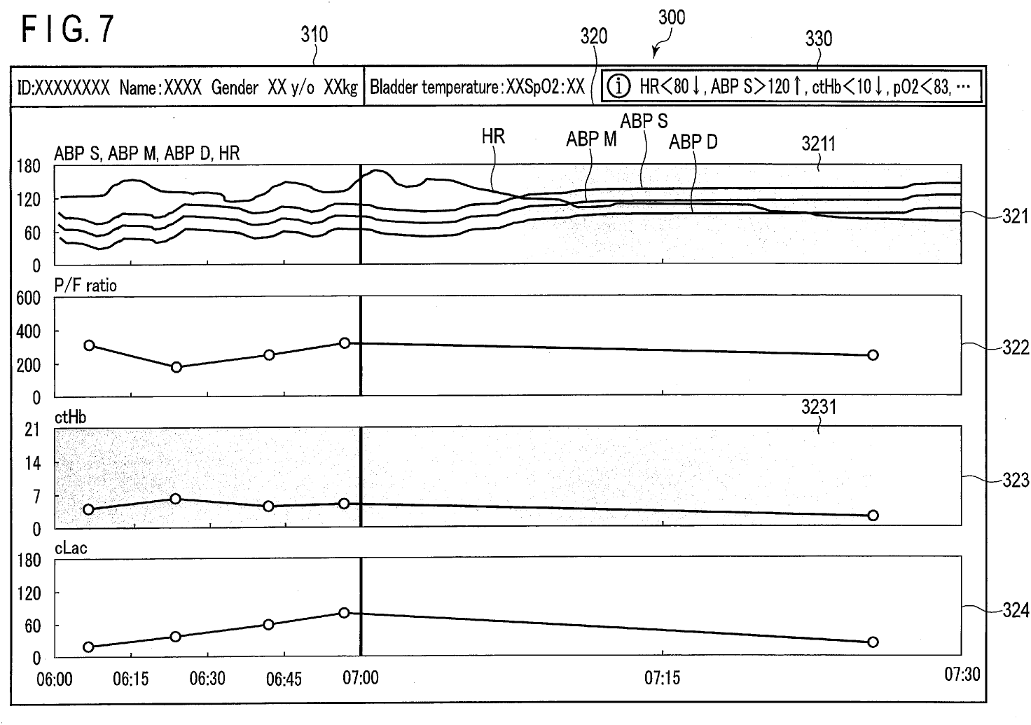

FIG. 7 is a view showing an example of the display screen 300 displayed on the display 23 through the emergency medical care support processing according to the present modification. In one example shown in FIG. 7, the alert information displayer 330 displays thereon a trend of change in addition to a name of an alert item and an alert condition for the alert item. For example, on the alert information displayer 330, the symbol “↓” shown for the heat rate (HR) indicates that its measurement value gently falls during a period in which the alert condition therefor is satisfied. Furthermore, the symbol “↑” shown for the upper blood pressure value (ABP S) indicates that its measurement value gently rises during a period in which the alert condition therefor is satisfied. As a method for displaying a trend of change, such a trend may be displayed by using another symbol different from that of FIG. 7, a color preset in accordance with the trend of change, or a character or sentence.

Hereinafter, the effect of the medical support system 100 according to the present modification will be described.

The medical support system 100 according to the present modification enables a trend of change in an alert value to be displayed on the display screen. For example, a trend of change in an alert item is displayed as the trend of change in an alert value. The above configuration enables a user such as an ER doctor to confirm the latest trend of change in medical data for which a warning has been given, thereby being able to use this information in making a decision in emergency medical treatment.

### Second Embodiment

Next, the second embodiment will be described. The present embodiment is a modification of the configuration of the first embodiment as will be described below. In the present embodiment, in the case where an alert has occurred in an item currently not displayed, this item is displayed in another window or dialog. Descriptions of the configurations, operations, and advantageous effects similar to those of the first embodiment will be omitted.

In the case where an alert value is not displayed on the display screen, the processing circuit 25 of the client device 20 causes, through the display control function 252, the display screen to display medical data in which the alert value has been detected. Specifically, in the case where items displayed on the display screen do not contain an alert item, the processing circuit 25 causes the display screen to display a change over time in the alert item and its measurement value. At this time, the processing circuit 25 displays a graph showing the change over time in the measurement value of the alert item in an overlapping manner on the display screen currently displayed.

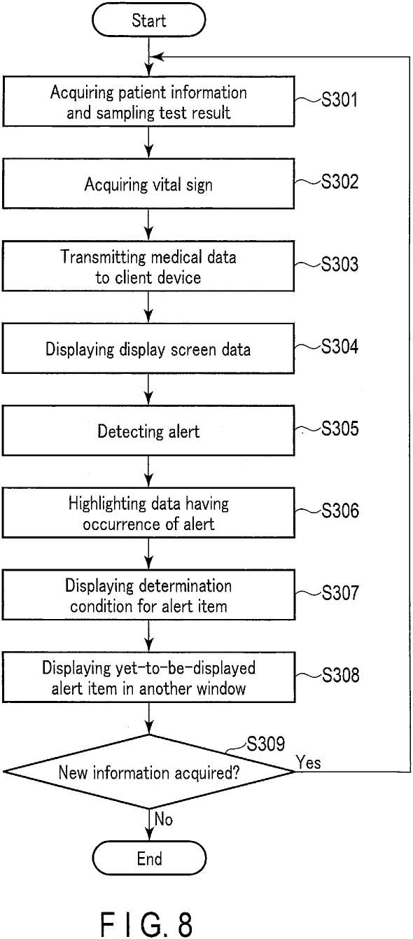

Next, the operation of the emergency medical care support processing executed by the medical support system 100 according to the present embodiment will be described. FIG. 8 is a flowchart showing an example of a processing procedure of emergency medical care support processing. Since the processing at steps S301 to S307 and S309 is the same as the processing at steps S101 to S108 in FIG. 4, descriptions thereof are omitted.

### (Emergency Medical Care Support Processing) (Step S308)

In the case where an alert item is not displayed on the current display screen, the processing circuit 25 of the client device 20 displays, through the display control function 252, a graph showing a change over time in the measurement value of the alert item in an overlapping manner on the display screen currently displayed.

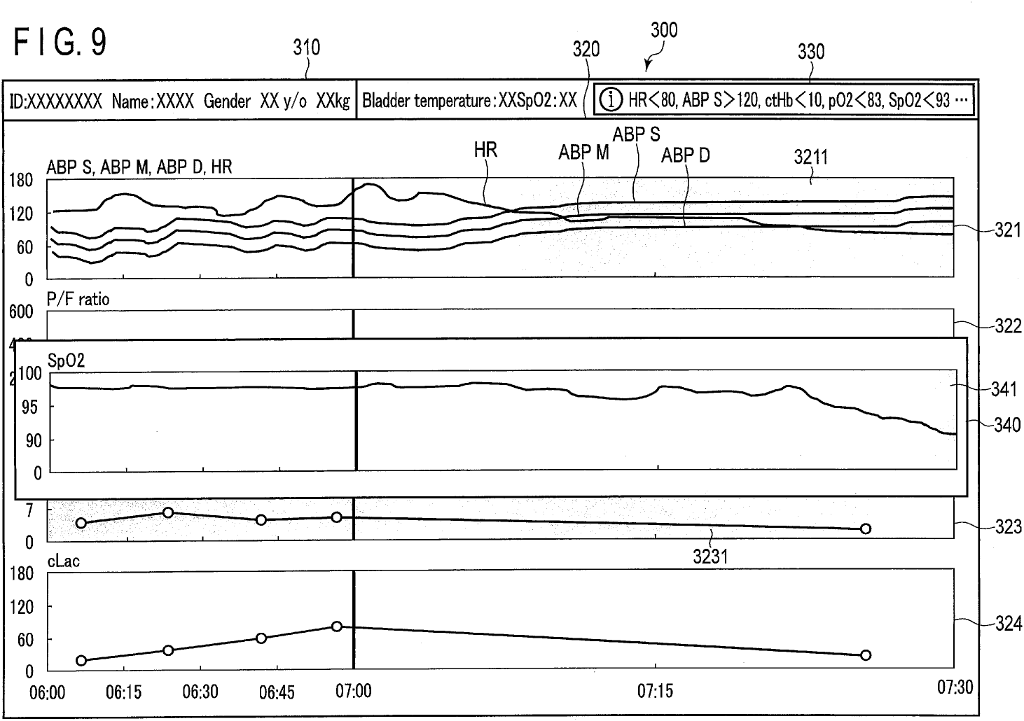

FIG. 9 is a view showing an example of the display screen 300 displayed on the display 23 through the emergency medical care support processing according to the present embodiment. FIG. 9 shows an example in which a warning data displayer 340 displays a graph showing a change over time in oxygen saturation (SpO2) detected as an alert item in a case in which the oxygen saturation (SpO2) is not displayed on the medical data displayer 320. The warning data displayer 340 is displayed in an overlapping manner on the medical data displayer 320. On the warning data displayer 340, a warning area 341 in which an alarm value which is a measurement value smaller than 93 is displayed is highlighted.

A display position of the warning data displayer 340 on the display screen 300 may be set by a user at a given position or may be switched by operation performed while the warning data displayer 340 is displayed. The warning data displayer 340 may be displayed in a different window than the display screen 300 or may be pop-up displayed on the display screen 300. In the case where there are a plurality of alert items that are not displayed on the display screen 300, all of them may be displayed, or a plurality of warning data displayers 340 may display them one at a time. The warning data displayer 340 may be displayed for a certain period of time or may be displayed until a user’s operation is received.

The warning data displayer 340 may be displayed only in the case where a period of time during which an alert condition is satisfied exceeds a predetermined period of time or may be displayed only in the case where the number of measurement values that satisfy the alert condition exceeds a predetermined number.

Hereinafter, the effect of the medical support system 100 according to the present embodiment will be described.

The medical support system 100 according to the present embodiment enables the display screen to display, in the case where an alert value is not displayed on the display screen, medical data in which the alert value has been detected. With this configuration, even in the case where the alert item is not displayed on the display screen, a user such as an ER doctor can easily confirm, without performing any special operations, a change over time in a measurement value in an item in which the alert has occurred. Accordingly, the user can confirm the latest trend of change in medical data for which the warning has been given, thereby being able to use this information for the decision in emergency medical treatment.

### Modification of Second Embodiment

A modification of the second embodiment will be described. The present modification is a modification of the configuration of the second embodiment as will be described below. In the present embodiment, in the case where an alert has occurred in an item currently not displayed, this item is displayed by replacing an item currently displayed. Descriptions of the configurations, operations, and advantageous effects similar to those of the second embodiment will be omitted.

In the case where an alert value is not displayed on the display screen, the processing circuit 25 of the client device 20 causes, through the display control function 252, the display screen to display, in place of medical data displayed thereon, medical data in which the alert value has been detected. Specifically, in the case where items displayed on the display screen do not contain an alert item, the processing circuit 25 causes the display screen to display, in place of the items displayed thereon, a change over time in an alert item. At this time, the processing circuit 25 displays a graph showing the change over time in a measurement value of the alert item by replacing the items currently displayed with the graph.

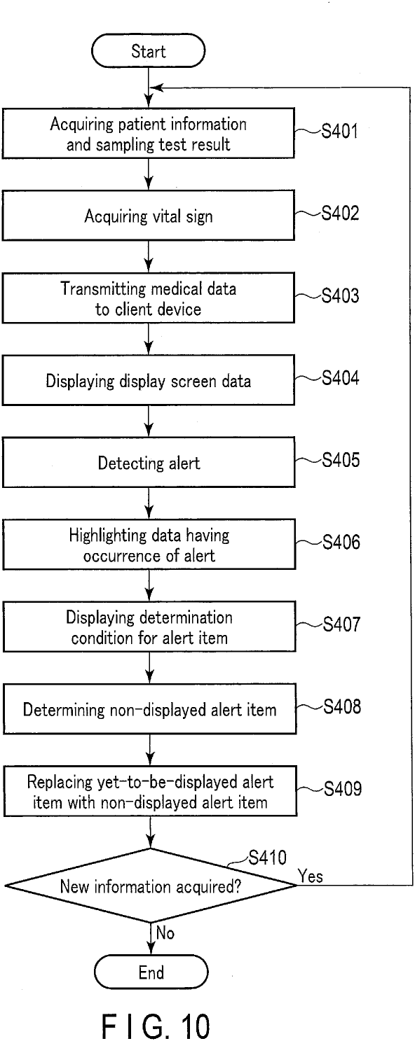

Next, the operation of the emergency medical care support processing executed by the medical support system 100 according to the present modification will be described. FIG. 10 is a flowchart showing an example of a processing procedure of emergency medical care support processing. Since the processing at steps S401 to S407 and S410 is the same as the processing at steps S301 to S307 and S309 in FIG. 8, descriptions thereof are omitted.

### (Emergency Medical Care Support Processing) (Step S408)

In the case where an alert item is not displayed on the current display screen, the processing circuit 25 of the client device 20 determines, through the display control function 252, a non-displayed item to be deleted from the display screen. For example, an item for which an alert has not occurred recently or an item in which the latest alert time is farthest from the present is selected as the non-displayed item from the items currently displayed.

### (Step S409)

Next, the processing circuit 25 replaces, through the display control function 252, an alert item not displayed yet (yet-to-be-displayed item) with a non-displayed item. Specifically, the processing circuit 25 displays a graph showing a change over time in measurement value of an alert item, instead of the non-displayed item determined at step S408.

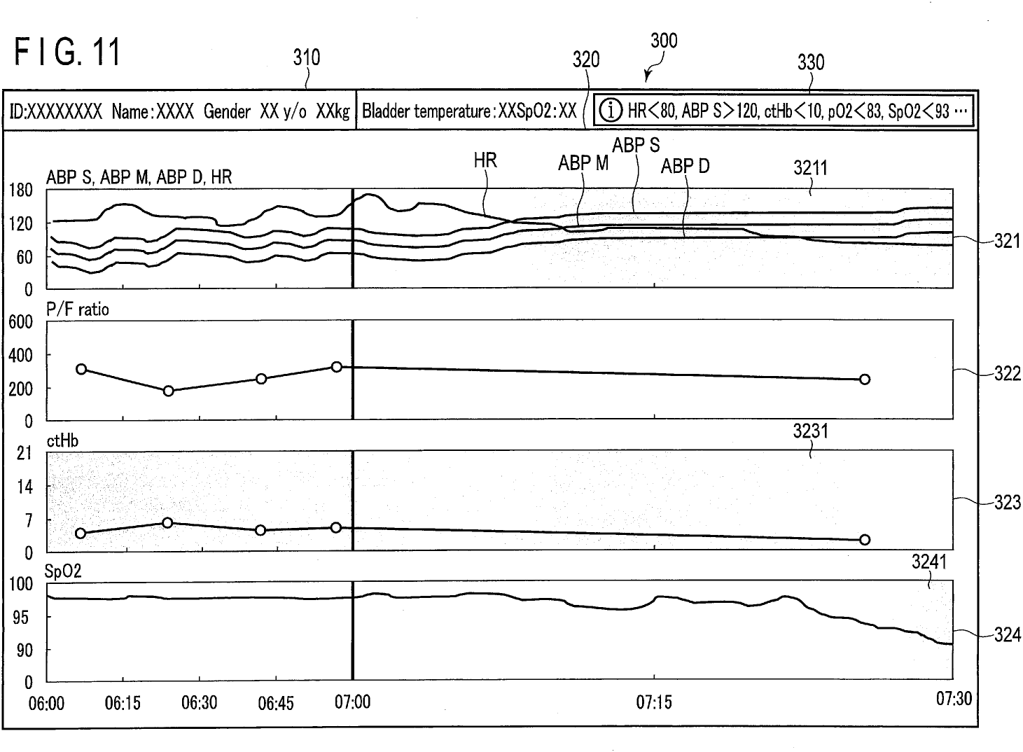

FIG. 11 is a view showing an example of the display screen 300 displayed on the display 23 through the emergency medical care support processing according to the present modification. In FIG. 11, in a case in which oxygen saturation (SpO2) detected as the alert item is not displayed on the medical data displayer 320, a fourth displayer 324 is caused to display a graph showing a change over time in the oxygen saturation (SpO2), instead of a graph showing a change over time in a lactate level (cLac). On the fourth displayer 324, a warning area 3241 displaying an alarm value that is a measurement value smaller than 93 is highlighted.

Hereinafter, the effect of the medical support system 100 according to the present modification will be described.

The medical support system 100 according to the present modification enables the display screen to display, in the case where an alert value is not displayed on the display screen, medical data in which the alert value has been detected, in place of medical data currently displayed on the display screen. This configuration enables more important information to be displayed with higher priority on the display screen.

### Third Embodiment

Next, the third embodiment will be described. The present embodiment is a modification of the configuration of the first embodiment as will be described below. In the present embodiment, in the case where specific data is newly acquired, a dialog or another window reporting this fact is automatically displayed. Descriptions of the configurations, operations, and advantageous effects similar to those of the first embodiment will be omitted.

Every time specific medical data is newly acquired, the processing circuit 25 of the client device 20 causes, through the display control function 252, the display screen to display the medical data newly acquired. For example, every time a sampling test result is newly acquired, the processing circuit 25 displays the acquired sampling test result for a certain period of time on the display screen of the display 23.

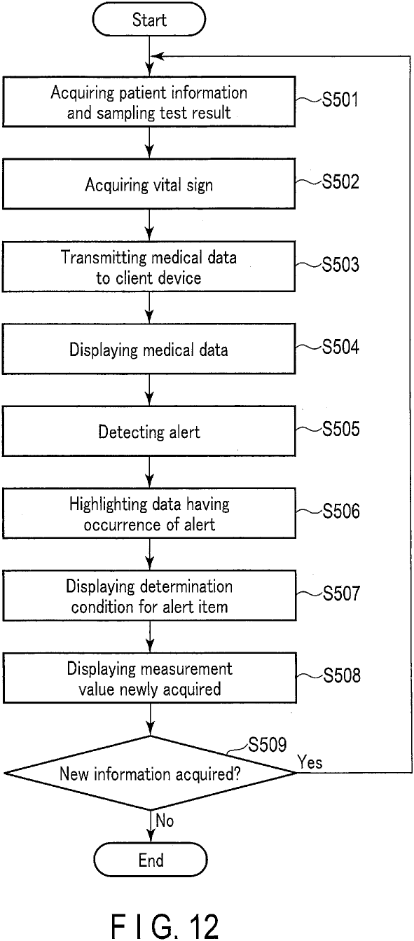

Next, the operation of the emergency medical care support processing executed by the medical support system 100 according to the present embodiment will be described. FIG. 12 is a flowchart showing an example of a processing procedure of emergency medical care support processing. Since the processing at steps S501 to S507 and S509 is the same as the processing at steps S101 to S108 in FIG. 2, descriptions thereof are omitted.

### (Emergency Medical Care Support Processing) (Step S508)

In a case where a sampling test result is newly acquired, the processing circuit 25 causes the display screen displayed on the display 23 to display a measurement value of the sampling test result newly acquired.

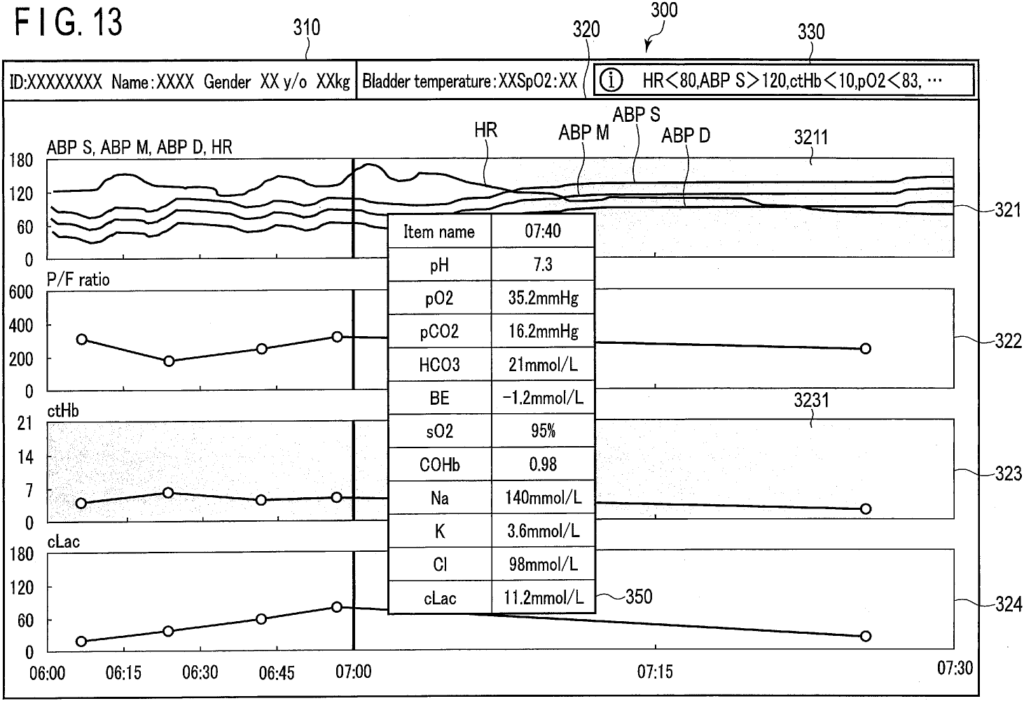

FIG. 13 is a view showing an example of the display screen 300 displayed on the display 23 through the emergency medical care support processing according to the present embodiment. In FIG. 13, the display screen 300 includes an update notifier 350. The update notifier 350 is displayed in an overlapping manner on the medical data displayer 320 in the case where a sampling test result is newly acquired. The update notifier 350 displays thereon the sampling test result newly acquired.

A display position of the update notifier 350 on the display screen 300 may be set by a user at a given position or may be switched by an operation performed while the update notifier 350 is displayed. Furthermore, the sampling test result newly acquired may not only be displayed on the screen but also be recited by voice.

Hereinafter, the effect of the medical support system 100 according to the present embodiment will be described.

The medical support system 100 according to the present embodiment enables, every time specific medical data is newly acquired, this data to be displayed on the display screen. For example, every time a sampling test result is updated, the updated sampling test result is displayed for a certain period of time. The above configuration enables, in a case where a sampling test result is updated, a user such as an ER doctor to easily confirm the updated sampling test result without making any special operation, thereby being able to use this information for making a decision in emergency medical treatment.

### Modification of Third Embodiment

A modification of the third embodiment will be described. The present modification is a modification of the configuration of the third embodiment as will be described below. Descriptions of the configurations, operations, and advantageous effects similar to those of the third embodiment will be omitted.

In a case where specific medical data is newly acquired, the processing circuit 25 of the client device 20 causes, through the display control function 252, the display screen to display the medical data newly acquired. At this time, in a case where the medical data newly acquired contains an alert value, this alert value is highlighted. The alert value is highlighted by changing, for example, a character in terms of a color, a size, a font, etc., or by changing a background in terms of a color, a pattern, etc. Furthermore, in a case where medical data newly acquired contains an alert value, the processing circuit 25 displays not only the alert value but also a past measurement value for a corresponding alert item.

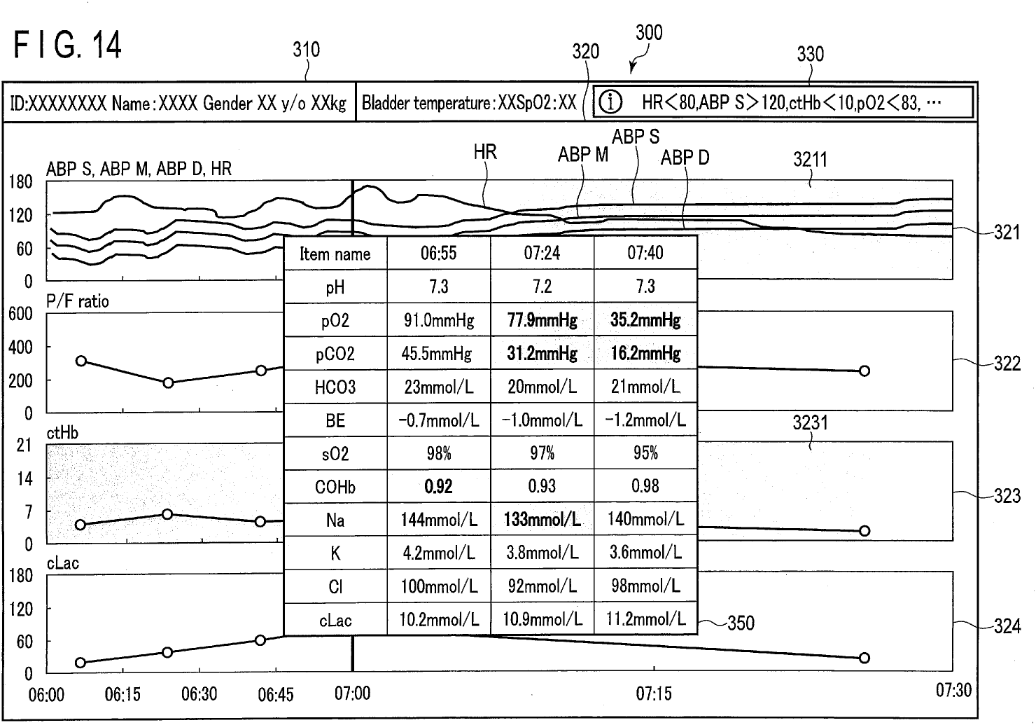

FIG. 14 is a view showing an example of the display screen 300 displayed on the display 23 through the emergency medical care support processing according to the present modification. In FIG. 14, the alert values are highlighted in bold type with the background in a different color from those of the remaining portions. In FIG. 14, since the latest sampling test result contains the alert values, not only the latest measurement values in the sampling test result but also the measurement values in the past two sampling test results are displayed.

The number of past measurement values to be displayed may be preset or may be switched by operations performed while they are displayed. With respect to the alert items, the measurement values may be recited by voice, and also the occurrence of an alert may be reported by voice. Alternatively, only the measurement values for the alert item may be recited by voice.

The above configuration enables, in a case where a sampling test result is updated, a user such as an ER doctor to easily know an item that he or she should keep in mind. By confirming not only current measurement values but also past measurement values for the item to keep in mind, information on the past measurement values can be used in making a decision in emergency medical treatment.

According to at least one embodiment described above, the occurrence of an alert can be efficiently given in emergency medical care.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical support system comprising a processing circuit configured to:
acquire medical data on a patient over time;
cause a display to display a display screen on which one or more measurement values in the medical data are displayed;
detect an alert value that satisfies a determination condition from the one or more measurement values included in the medical data every time the medical data is acquired; and
highlight an area in which the alert value is displayed on the display screen, and cause the display screen to display information on the determination condition for all detected alert values.

2. The medical support system according to claim 1, wherein the information on the determination condition includes the alert value, a conditional expression for the alert value, or a name of an item of the alert value.

3. The medical support system according to claim 1, wherein the processing circuit is further configured to cause the display screen to display a trend of change in the alert value.

4. The medical support system according to claim 1, wherein in a case where the display screen does not display the alert value, the processing circuit is further configured to cause the display screen to display medical data from which the alert value has been detected.

5. The medical support system according to claim 1, wherein in a case where the display screen does not display the alert value, the processing circuit is further configured to cause the display screen to display medical data from which the alert value has been detected, in place of medical data currently displayed on the display screen.

6. The medical support system according to claim 1, wherein every time specific medical data is newly acquired, the processing circuit is further configured to cause the display screen to display the medical data newly acquired.
